(19) United States
(12) Patent Application Publication
(10) Pub. No.: US 2024/0424462 A1
(43) Pub. Date: Dec. 26, 2024

(54) FIXED-BED TUBULAR REACTOR COMPRISING A MAKE-UP CHAMBER

(71) Applicant: COMMISSARIAT A L'ENERGIE ATOMIQUE ET AUX ENERGIES ALTERNATIVES, Paris (FR)

(72) Inventors: Frédéric DUCROS, Grenoble Cedex 09 (FR); Isabelle CHAMPON, Grenoble Cedex 09 (FR); Albin CHAISE, Grenoble Cedex 09 (FR); Laurent BEDEL, Grenoble Cedex 09 (FR)

(73) Assignee: COMMISSARIAT A L'ENERGIE ATOMIQUE ET AUX ENERGIES ALTERNATIVES, Paris (FR)

(21) Appl. No.: 18/698,273

(22) PCT Filed: Oct. 3, 2022
(86) PCT No.: PCT/FR2022/051862

§ 371 Date: Apr. 3, 2024

(30) Foreign Application Priority Data
Oct. 5, 2021 (FR) FR2110518

(51) Int. Cl. B01J 8/02; B01J 8/06; C07C 1/12

(52) U.S. Cl. CPC B01J 8/0214; B01J 8/065; B01J 8/067; C07C 1/12; B01J 2208/00884; B01J 2208/00938; B01J 2208/065

(57) ABSTRACT

A fixed-bed tubular reactor that extends between first and second ends and includes a bed of catalyst powder confined in an annular space defined by an outer wall of a hollow tube and an inner wall of a hollow insert, which comprises a distribution chamber and a collection chamber. The tubular reactor comprises at least one make-up chamber separated from the at least one distribution chamber and the at least one collection chamber by at least one first dividing wall. The at least one make-up chamber has an inlet port for at least one make-up fluid, which differs from the reactive gases, at the first end and an outlet port for the at least one make-up fluid at the second end.

### TECHNICAL FIELD

The present invention relates to the general field of exchange reactors, and more particularly to the field of catalytic exchange reactors using a solid catalyst, in particular in powder form, and intended for the implementation of endothermic or mainly exothermic catalytic reactions.

Such reactions can in particular be implemented for the synthesis of fuels and combustibles, for example liquid combustibles, such as methanol (MeOH), or gas combustibles, such as methane or natural gas substitute (SNG for “Synthetic Natural Gas”), dimethyl ether (DME), hydrocarbons or olefins, obtained from hydrogen and carbon oxides or from synthesis gas comprising a mixture of hydrogen and carbon oxides. They may also relate to the generation of hydrogen from methane, the synthesis of ammonia from hydrogen and nitrogen, or related hydrogenation and dehydrogenation reactions between hydrogen and molecules of the liquid organic hydrogen carrier type (or LOHC for “Liquid Organic Hydrogen Carriers”).

The invention can thus be particularly applied to very exothermic reactions, such as that of methanation (or more generally the production of hydrocarbons or hydrogenation reactions) of carbon monoxide or dioxide in the presence of hydrogen. It can also be applied to reactions such as that of Fischer-Tropsch, that of wet or dry reforming of methane or other hydrocarbons, or else oxidation or dehydrogenation reactions. The invention can also be used as a heat exchanger for applications, in particular those using a gas, which require frequent maintenance operations, for example due to corrosion, clogging, among others.

The invention thus proposes a fixed-bed tubular reactor capable of implementing in particular exothermic organic synthesis methods.

### PRIOR ART

Catalytic reactors using solid catalysts are widely used for the synthesis of organic compounds such as synthetic fuels or combustibles including natural gas substitutes (SNG), dimethyl ether, methanol, hydrocarbons or else olefins.

In the context of the production of hydrocarbons, or more generally for hydrogenation reactions, from hydrogen and carbon oxide, the equilibria involved, or also called main reactions hereafter, are generically the following:

$$n CO + ( 2 n + 1 ) {H}_{2} ⇔ {C}_{n} {H}_{2 n + 2} + {n H}_{2} O \qquad Eq . 1$$

$${n CO}_{2} + ( 3 n + 1 ) {H}_{2} ⇔ {C}_{n} {H}_{2 n + 2} + 2 {n H}_{2} O \qquad Eq . 2$$

$$X + {n H}_{2} ⇔ ( - {X H}_{2 n} - ) \qquad Eq . 3$$

The equilibrium described by the third equation (Eq.3) relates to molecules of the liquid organic hydrogen carrier (LOHC) type. It is a reversible reaction which is therefore successively carried out in exothermic then endothermic sequences. These various reactions are widely documented on the thermochemical level. They potentially give rise to side reactions (for example of the WGS type (“Water Gas Shift”), RWGS (Reverse Water Gas Shift”), formation of Boudouard, etc.) known to the person skilled in the art and not described here.

The species concerned by these main reactions and side reactions are called “reactants” for the species entering the reactor, and “products” for the species produced by the main and ancillary reactions. In addition, “reactive fluids” describes all the species affected by these reactions. These reactions are all associated with thermodynamic conditions, pressure and temperature, which are preferential given the performance targets: it is generally interesting to work at high pressure and it is also necessary to correctly manage the exothermicity of these reactions by extracting heat of the reaction domain, which allows to guarantee the conversion rate, the selectivity, the lifespan of the catalysts, among others. Other features discussed later are also of interest.

Many architectures of catalytic reactors used in industry are already known to ensure the control and thermal management of endothermic or exothermic chemical reactions. The main types of exchange reactors known for exothermic reactions are described below.

First of all, the simplest catalytic reactor technology is the technology called “fixed bed” reactor technology. In adiabatic fixed bed reactors placed in cascade, exothermicity is then generally managed by dilution of the reagents at the inlet of the first reaction stage, for example by recirculation of the products, and by the installation of heat exchangers intended to cool the reactant-product mixture between the different reactors. This architecture has the advantage of simplicity of manufacturing but requires the implementation of gas recycles to limit the temperature rise and requires the use of catalysts stable at high temperature. These reactors are rather used as centralized units operating at steady state.

There is also the technology of fluidised bed exchange reactors. Such reactors were developed to address the problem of heat transfer in fixed beds. These reactors offer the advantage of good thermal homogeneity in the reactor, which avoids hot spots but require, at equivalent power, a larger reactor volume than in the case of fixed plug-flow beds. In these reactors, the catalyst is in the form of fine particles whose attrition must be controlled. In addition, particle fluidisation requires limiting the gas flow variation ranges, which makes these reactors not very flexible with regard to intermittent operations.

Another exchange reactor technology relates to exchange reactors wherein the chemical reaction takes place within a reactive channel continuously cooled by an external heat transfer fluid. Most of these reactors are of the tube-and-shell type, the reaction occurring in the reaction tubes cooled at the periphery by a heat transfer bath. The reactive gases circulate axially in the tubes which contain a catalyst, for example in powder form.

The combination of exchange reactors of the same type or of different types within the same unit can also be considered in order to improve the conversion, flexibility or valorisation of the recovered heat.

Managing thermal constraints on reactors begins with responding to the need for thermal control. This can take various routes as explained in the previous technological solutions, namely outside the reactor solutions such as staging the overall conversion, with intermediate cooling and/or dilution, and/or condensation; solutions inside the reactor such as the evolution towards the concept of exchange reactors, the intensification of heat exchanges, the reduction in the sizes of the reactive channels (millistructuring), the integration of 3D conductive structures for thermal homogenisation, the staging of injections of reagents allowing to distribute the energy deposition.

As for the question of the lifespan of the catalysts, it should be noted that exothermic reactions generating a large amount of heat, this can lead to the appearance of hot spots resulting in local degradation of the catalyst and degradation of the conversion performance of the reactor/catalyst assembly. In other words, the degradation of the solid catalyst can result in a deactivation of the latter and lead to a reduction in the conversion rate of the chemical species present. The selectivity of the reactions involved is also affected.

Moreover, certain hydrogenation reactions very useful to industry, such as the synthesis of methanol or the Fischer-Tropsch synthesis, as described by the previous equations Eq.1 to Eq.3 are balanced and have, for thermodynamic conditions usually retained, low to moderate conversion rates. An interesting strategy then consists of associating permselective membranes with one or more walls of the reactive channels, so as to subtract a species produced, typically water, from the reactive medium, which unbalances the reaction and increases its productivity. The advantage of such a method could be demonstrated experimentally and theoretically. Innovations can relate to two main elements: membranes and methods of integration into reactors.

In the conventional solution of an exchange reactor with the catalyst located in the tubes, for tube-and-shell type technologies, one of the problems is the control of the zones made hotter due to the exothermicity of the reaction. This phenomenon requires the tubes to be intensely cooled over their entire surface while at a given time, only a small part of this surface needs to be cooled intensely, for example for injection and axial circulation of the reactants. A consequence of this is an oversizing of the thermal fluid flow.

In order to overcome these problems, provision was made of an arrangement allowing to distribute reactants over the entire length of the tubes. This solution then allows to obtain better temperature homogeneity over the entire length of the reactor. In this regard, documents U.S. Pat. Nos. 3,758,279 A, 4,374,094 A, EP 0 560 157 A1 and U.S. Pat. No. 2,997,374 A propose exchange reactors implementing distribution of reagents from an annular distribution space. In particular, these exchange reactors, which are generally cylindrical in shape, comprise, arranged coaxially and from the outside of the reactor, a tube, the annular distribution space, a catalyst charge and a collection space. These solutions constitute the standard for cooling in tube-and-shell type reactors, that is to say that the cooling of the tubes is controlled only by the flow in the shell.

However, these arrangements are not satisfactory. Indeed, the presence of the annular distribution space disposed around the catalyst charge limits heat transfer from the catalyst to the tube, making ineffective the cooling systems generally considered. It nevertheless remains possible to insert heat-conducting elements into the reactor. However, such a solution remains incompatible with reactors comprising small diameter tubes.

Conversely, document CN 103990420 A proposes to implement an insert provided with a distribution chamber and a collection chamber, disposed in the centre of a tube and defining therewith an annular space housing the solid catalyst. However, the arrangement proposed in this document does not allow homogeneous distribution of the temperature within the annular space. More particularly, this arrangement does not allow to obtain an optimal temperature profile within the solid catalyst.

FIG. 1 of document U.S. Pat. No. 8,961,909 A represents another example of a tube-and-shell type reactor. This reactor is in particular provided with an injection tube, immersed in a bed of catalytic powder, and along which holes are made. The latter are in particular arranged to ensure injection of the reactive gas at different levels of the bed of catalytic powder, and thus limit the appearance of hot spots in said bed. However, this reactor is not satisfactory. Indeed, in order to ensure its cooling, this reactor requires the installation of a plurality of circuits for circulating a heat transfer fluid, which further increases its complexity.

Document U.S. Pat. No. 7,402,719 B2, in particular in FIG. 3a, discloses another example of a reactor arranged to allow a staged injection of a reactant C with a view to its reaction with a reactant A. This reactor comprises in this regard two layers (or channels) separated by a wall and intended to ensure the circulation, respectively, of reactant A and reactant C. The two layers are also in fluid communication by means of a plurality of holes made in the wall separating them. These holes are arranged in particular to ensure progressive mixing of reactant C with reactant A. This progressive mixing thus allows to limit the appearance of hot spots. However, the arrangement of the reactor in the form of a stack of sheets makes it not very compact.

Furthermore, in patent application FR 3 103 714 A1 of the Applicant, an insert type device organises an axial distribution of the reactive gases in the tubes, a circulation of gases in the substantially orthoradial direction through a bed of catalyst in an annular space defined by the internal wall of a tube and the external wall of the insert, and an axial collection of the gases produced.

There is still a need to improve these types of reactors, and in particular to provide such tube/shell reactors with alternative fluid circulation allowing to improve cooling in particular.

### DESCRIPTION OF THE INVENTION

The invention therefore aims at least partially at overcoming the needs mentioned above and the disadvantages relating to the achievements of the prior art.

The invention aims in particular at proposing a fixed-bed tubular reactor allowing a more uniform distribution of the reactants within the solid catalyst, a more homogeneous distribution of the heat flow generated within the solid catalyst, a better cooling management. In addition, the invention seeks to propose a tubular reactor for which the reliability and lifespan (of the catalysts) are improved compared to reactors known from the prior art, and allowing to optimise (increase) the passage time of gases in the fixed bed of catalytic powder.

The invention thus has, according to one of its aspects, a fixed-bed tubular reactor which extends, along a longitudinal axis, between a first end and a second end, said reactor comprising a bed of catalytic powder confined in an annular space delimited by an external wall of a hollow tube and an internal wall of a hollow insert disposed coaxially in the hollow tube, the hollow insert comprising at least one distribution chamber and at least one collection chamber, separated from each other by at least one first separating wall, said at least one distribution chamber and said at least one collection chamber comprising, respectively, a gas admission opening at the first end and a gas evacuation opening at the second end,
characterised in that the reactor further includes at least one make-up chamber separated from said at least one distribution chamber and from said at least one collection chamber by at least one first separating wall, said at least one make-up chamber comprising an inlet orifice for at least one make-up fluid, distinct from the gases circulating in said at least one distribution chamber and in said at least one collection chamber, at the first end and an outlet orifice of said at least one make-up fluid at the second end.

Thanks to the invention, it is in particular possible to improve the cooling of the reaction zone (reactants, reaction products and catalysts) by allowing this zone to be cooled by internal and external circulation of heat transfer fluid, which is beneficial for all hydrogenation reactions for example. The overall performance of the reactor (conversion rate and selectivity of reactions) will be improved by better temperature homogeneity resulting from the application of the invention.

The reactor according to the invention may further include one or more of the following features taken individually or in any possible technical combination.

Each of said at least one distribution chamber, at least one collection chamber and at least one make-up chamber can be delimited by a section of the internal wall and two first separating walls.

In addition, the reactor may include at the first end and at the second end, respectively, a distributor space and a collector space between which the hollow insert is disposed.

Furthermore, the tube and the insert can be held by at least two tubular holding plates respectively at the first and second ends, at least one of said at least two tubular holding plates, in particular all the tubular holding plates, including an inlet or outlet conduit respectively fluidly connected to a make-up fluid inlet or outlet orifice and formed in said at least one of said at least two tubular holding plates, in particular to allow lateral admission and/or extraction of make-up fluid.

The axial lengths of the tube and the insert may be different, the axial length of the tube being in particular shorter than the axial length of the insert.

Alternatively, the tube and the insert can be held by at least two tubular holding plates respectively at the first and second ends, and at least one inlet or outlet conduit respectively fluidly connected to a make-up fluid inlet or outlet orifice can be located respectively in the distributor space and in the collector space, outside said at least two tubular holding plates, to allow in particular lateral admission and/or extraction of make-up fluid.

Further alternatively, the tube and the insert can be held by at least two tubular holding plates respectively at the first and second ends, and said inlet orifice and said outlet orifice of said at least one make-up fluid can be respectively formed in the upper and lower ends of the insert to respectively allow a supply from the distributor space and an extraction from the collector space, the reactor further comprising at least one lateral gas supply conduit and at least one lateral gas extraction conduit.

Said at least one lateral supply conduit and the supply of make-up fluid to the distributor space on the one hand, and said at least one lateral extraction conduit and the extraction of make-up fluid from the collector space on the other hand, can be separated by a sealed separation plate.

Furthermore, a seal can be disposed between the insert and each of the sealed separation plates.

Moreover, the flow of said at least one make-up fluid can be carried out in a co-current or counter-current manner to the flow of gases in said at least one distribution and collection chamber.

If necessary, the catalytic powder can be retained in the annular space by a fibrous material seal at each of the ends of the annular space.

Advantageously, said at least one make-up fluid can be a heat transfer fluid, in particular a cooling fluid.

In addition, the insert may be a one-piece part.

Moreover, the invention also relates, according to another of its objects, to the use of a tubular reactor as defined above, characterised in that it implements endothermic or exothermic reactions for the synthesis of fuels and of combustibles.

In particular, the annular space can allow a hydrogenation reaction and said at least one make-up chamber can allow a dehydrogenation reaction, or vice versa.

### BRIEF DESCRIPTION OF THE DRAWINGS

The invention can be better understood upon reading the detailed description which follows, non-limiting examples of its implementation, as well as upon examining the schematic and partial figures of the appended drawing, on which:

FIG. 1 is a schematic representation of a fixed bed tubular reactor in accordance with to the invention, according to a sectional plane, passing through the longitudinal axis of the reactor, in particular according to the longitudinal sectional plane PP of FIG. 2, allowing to visualise a collection chamber and a distribution chamber,

FIG. 2 is a sectional view along a transverse plane, or normal section, perpendicular to the longitudinal axis of the tubular reactor of FIG. 1,

FIG. 3 is a schematic representation of the tubular reactor of FIGS. 1 and 2 according to the longitudinal section plane P′P′ of FIG. 2, allowing to visualise two make-up chambers, supplied by lateral inlet and outlet orifices,

FIG. 4 is a view similar to that of FIG. 1 allowing to illustrate the use of seals to retain the catalyst,

FIG. 5 is a view similar to that of FIG. 3 allowing to illustrate the use of seals to retain the catalyst,

FIG. 6 is a schematic representation of another example of a tubular reactor in accordance with the invention, in a view similar to that of FIG. 1,

FIG. 7 is a view similar to that of FIG. 3 for the reactor of FIG. 6,

FIG. 8 is yet a schematic representation of another example of a tubular reactor in accordance with the invention, in a view similar to that of FIG. 1,

FIG. 9 is a view similar to that of FIG. 3 for the reactor of FIG. 8,

FIGS. 10 and 11 are views, in axial section respectively along the planes PP and P′P′ with reference to FIG. 2, of a plurality of tubular reactors similar to that of FIGS. 1 to 5, located inside a shell, and

FIG. 12 is a view, in axial section along the plane P′P′ with reference to FIG. 2, of a plurality of tubular reactors similar to that of FIGS. 6 and 7, located inside a shell.

In all these figures, identical references can designate identical or similar elements.

In addition, the different parts represented in the figures are not necessarily on a uniform scale, to make the figures more readable.

### DETAILED DESCRIPTION OF PARTICULAR EMBODIMENTS

The present invention relates to a tubular exchange reactor with a fixed catalytic powder bed. In particular, the bed of catalytic powder is confined in an annular space delimited by a wall, called the external wall, of a hollow tube and another wall, called the internal wall, of a hollow insert housed coaxially in said tube. The catalytic powder bed may in particular include a catalyst in the form of grains.

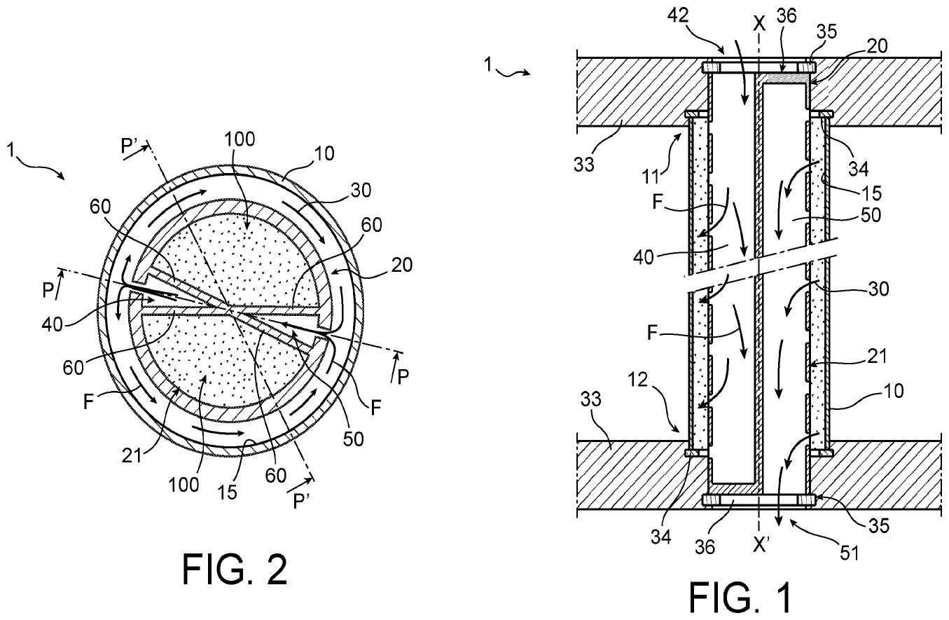

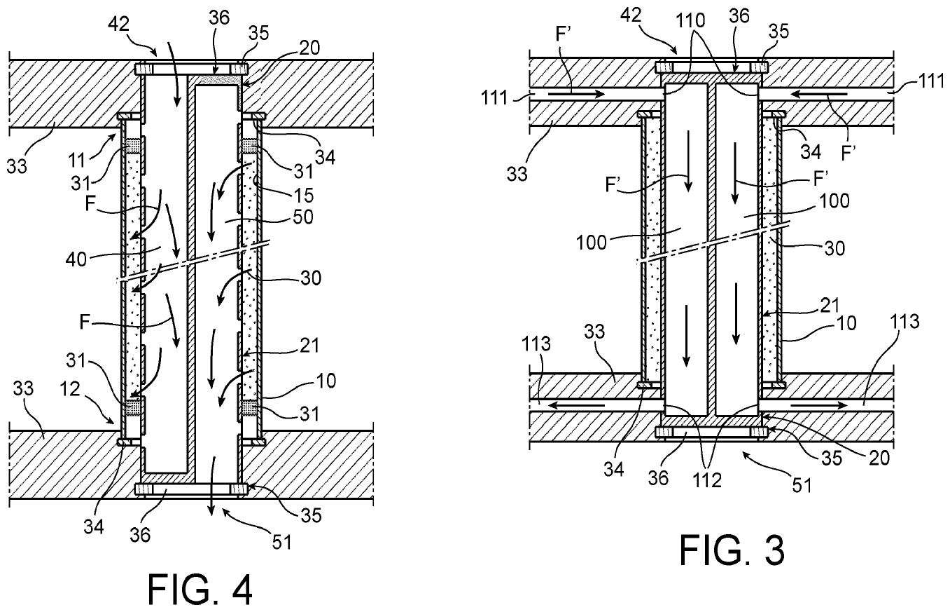

Thus, an exemplary embodiment of a fixed bed tubular reactor according to the present invention can be seen in FIGS. 1 and 2. It should be noted that in these FIGS. 1 and 2, as well as in all the figures described subsequently, the arrows F represent the direction of travel of the gas. Furthermore, the arrows F′, in particular visible in FIGS. 3, 5, 7, 9 and 11, represent the direction of travel of the make-up fluid.

The tubular reactor 1 according to the present invention comprises an external hollow tube 10 which extends along a longitudinal axis XX′ between a first end 11 and a second end 12. The hollow tube 10 can have a symmetry of revolution around the longitudinal axis XX′. It is therefore understood that the longitudinal axis XX′ can be an axis of revolution of the hollow tube 10.

The hollow tube 10 may comprise a metal, and in particular a metal selected from: steel, aluminium alloy, copper, nickel, among others. The diameter of the hollow tube 10 can be comprised between 5 mm and 100 mm. The wall, called the external wall 15, forming the hollow tube 10 may have a thickness comprised between 0.5 mm and 10 mm. The hollow tube 10 can have a length comprised between 10 times and 200 times the diameter of the internal surface.

The tubular reactor 1 also comprises a hollow insert 20 which also extends along the longitudinal axis XX′ and has a generally cylindrical shape. The hollow insert 20 is in particular housed in the volume of the hollow tube 10 coaxially with the latter. In particular, the insert 20 also comprises a wall, called the internal wall 21, in particular a wall possibly permeable to gas, which delimits with the external wall 15 an annular space 30. The annular space 30 is, in this respect, filled with a catalytic powder, and will be the site of the conversion reactions of reactive gases capable of passing through the tubular reactor 1. The annular space 30 can have a thickness, defined as the distance between the external wall 15 and the internal wall 21, comprised between 2% and 20% of the diameter of the internal surface of the hollow tube 10. The hollow insert 20 can be a one-piece part. The hollow insert 20 can for example be made of stainless steel, in particular by brazing methods, or of aluminium, in particular by extrusion or additive manufacturing methods (for example a 3D manufacturing method), or even of polymers for certain reactions at low temperatures. The various openings of the hollow insert 20 can be made during the manufacture of the insert, and/or made by machining in a second step.

The hollow insert 20 further comprises at least one distribution chamber 40 and at least one collection chamber 50. Here, for the sake of simplicity, a single distribution chamber 40 with a single injection point and a single collection chamber 50 are represented but this choice is not limiting. In particular, the hollow insert 20 may comprise in particular between 1 and 4 distribution chambers 40, and between 1 and 4 collection chambers 50.

Said at least one distribution chamber 40 and said at least one collection chamber 50 are advantageously disposed alternately, and extend over the entire length of the hollow insert 20.

Advantageously, the hollow insert 20 further comprises at least one make-up chamber 100 organising the axial circulation of a make-up fluid, here two make-up chambers 100, but the invention is not limited on the number of make-up chambers and make-up fluids.

Thus, a first make-up chamber 100, said at least one collection chamber 50, a second make-up chamber 100 and said at least one distribution chamber 40 are found successively by observation of a plane in section normal to the axis XX′ of the hollow tube 10, as visible in FIG. 2. Said at least one collection chamber 50, said at least one distribution chamber 40 and the make-up chambers 100 are also separated from each other by first separating walls 60. It is therefore understood that a distribution chamber 40 is delimited by two separating walls 60 and a section of the internal wall 21. In an equivalent manner, a collection chamber 50 is also delimited by two first separating walls 60 and another section of the internal wall 21. In a still equivalent manner, a make-up chamber 100 is separated by two first separating walls 60 and yet another section of the internal wall 21.

Furthermore, the first separating walls 60 extend over the entire length of the hollow insert 20 in the volume defined by the hollow tube 10, and are arranged to prevent any direct passage of gas from one chamber to the other. For example, the first separating walls 60 form planes passing through the longitudinal axis XX′.

In particular, the first two separating walls 60 of a distribution chamber 40 may have a generally elongated shape and extend along the longitudinal axis XX′ from the first end 11 towards the second end 12. In particular, the first two separating walls 60 of a distribution chamber 40 can have a common side coincident with the longitudinal axis XX′.

Furthermore, the reactor 1 may include, at the first end 11 of the hollow insert 20, a distributor space 42, or inlet plenum, through which one or more reactive gases are likely to be admitted into the distribution chamber 40 through an admission opening. Likewise, the reactor 1 may include, at the second end 12 of the hollow insert 20, a collector space 51, or outlet plenum, through which one or more gases are capable of being evacuated through an evacuation opening.

Moreover, the distribution chamber 40 is closed at the second end 12, and the collection chamber 50 is closed at the first end 11.

The internal wall 21 may further comprise at least one distributing opening and at least one collecting opening, respectively allowing the distribution of a gas capable of being admitted through the admission opening at the inlet plenum 42 in a distribution compartment towards the annular space 30, and the collection of the gas distributed in the annular space 30 by the collection chamber 50.

The hollow tube 10 is advantageously held by two tubular holding plates 33 which each include a first sealed system 34 for fixing the hollow tube 10 to each holding plate 33. Likewise, the hollow insert 20 is advantageously held by the two holding plates 33 which each include a second sealed system 35 for fixing the hollow insert 20 to each holding plate 33. Separating walls 36 are also present in each of the holding plates 33, between which the hollow tube 10 is contained and the hollow insert 20.

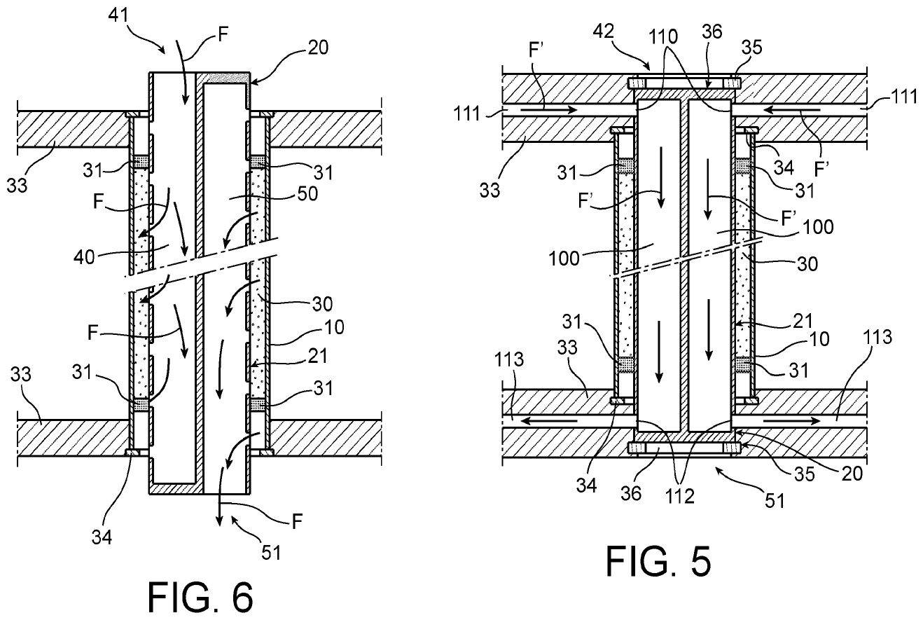

According to an advantageous aspect illustrated in FIGS. 4 and 5, the catalytic powder is retained in the annular space 30 by a seal 31, for example made of fibrous material, at each of the ends of the annular space 30. To the extent that the seal 31 is made of fibrous material, the latter is necessarily porous and therefore permeable to reactive gases. The fibrous material may in this regard comprise at least one of the elements selected from: glass fibre, ceramic fibre, metal fibre, carbon fibre, polymer material fibre.

The seal 31 may in particular be in the form of a braid, a sheath, a cord or simply comprise a filling of the fibrous material. The fibrous material is advantageously a thermal insulator and has a thermal conductivity substantially equivalent to that of the catalyst used (0.2 W/m/K to 10 W/m/K).

FIG. 3 allows to visualise, in axial section along the plane P′P′ of FIG. 2, the make-up chambers 100 described previously. Each make-up chamber 100 is supplied by a lateral inlet orifice 110, located near the end 11 of the hollow tube 10, this inlet orifice 110 being supplied by a lateral inlet conduit 111 formed in the upper holding plate 33.

Likewise, each make-up chamber 100 includes a lateral outlet orifice 112, located near the end 12 of the hollow tube 10, this outlet orifice 112 being fluidly connected to a lateral outlet conduit 113 formed in the lower holding plate 33. In this way, a make-up fluid F′ can be admitted by circulation into the tubular holding plates 33.

Advantageously, the invention thus allows to extend the thermalisation capacities of the reactor 1, in particular to allow to manage and circulate make-up fluids, in particular utility or reactive fluids, in addition to the reaction reactants and products, for example according to the equations Eq. 1 and/or Eq. 2 described previously.

The geometry of the hollow insert 20 and the geometry of the hollow external tube 10 are defined so as to allow a separate supply of the distribution 40 and make-up 100 chambers, and separate outlets of the collection 50 and make-up 100 chambers.

In the exemplary embodiment of FIGS. 1 to 5, the hollow tube 10 and the hollow insert 20 have different axial lengths, the hollow tube 10 being shorter than the hollow insert 20, and the make-up fluid supply and evacuation is done through a holding plate 33, via conduits 111 and 113. In addition, the flow of the make-up fluid F′ is directed axially and co-currently from the circulation of gases. However, a counter-current concept can also be interesting.

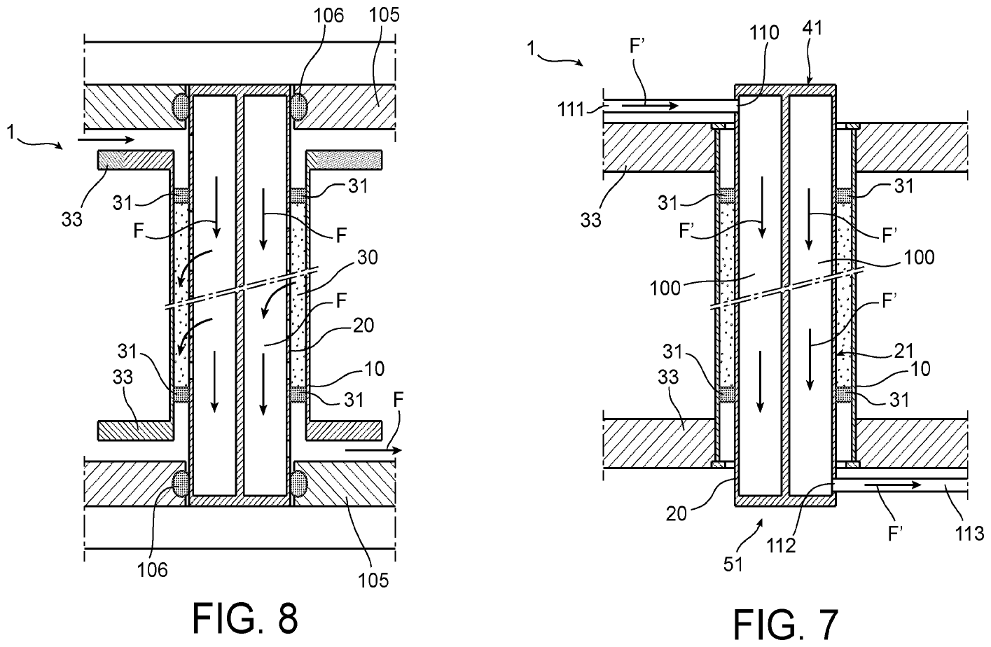

In the exemplary embodiment of FIGS. 6 and 7, the supply of the make-up chambers 100 is ensured by means of dedicated conduits located in the inlet 42 and outlet 51 plenums, and no longer by means of conduits machined in holding plates 33.

More precisely, as visible in FIG. 7, a lateral inlet conduit 111, for the admission of make-up fluid F′, is located along the upper holding plate 33, outside thereof, in the upper part of the reactor 1. Likewise, a lateral outlet conduit 112, for the extraction of make-up fluid F′, is located along the lower holding plate 33, outside thereof, in the lower part of the reactor 1.

Thus, the supplies and evacuations of the make-up fluid F′ are ensured by conduits 111, 112 made outside the tubular holding plates 33, and ensuring a tight connection with the exterior of the inlet 41 and outlet 51 plenums. In addition, the flow of the make-up fluid F′ is directed axially and co-currently with the circulation of gases. However, a counter-current concept can also be interesting.

In the exemplary embodiment of FIGS. 7 and 8, the supply of the make-up chambers 100 is ensured by means of additional connections in the inlet 42 and outlet 51 plenums.

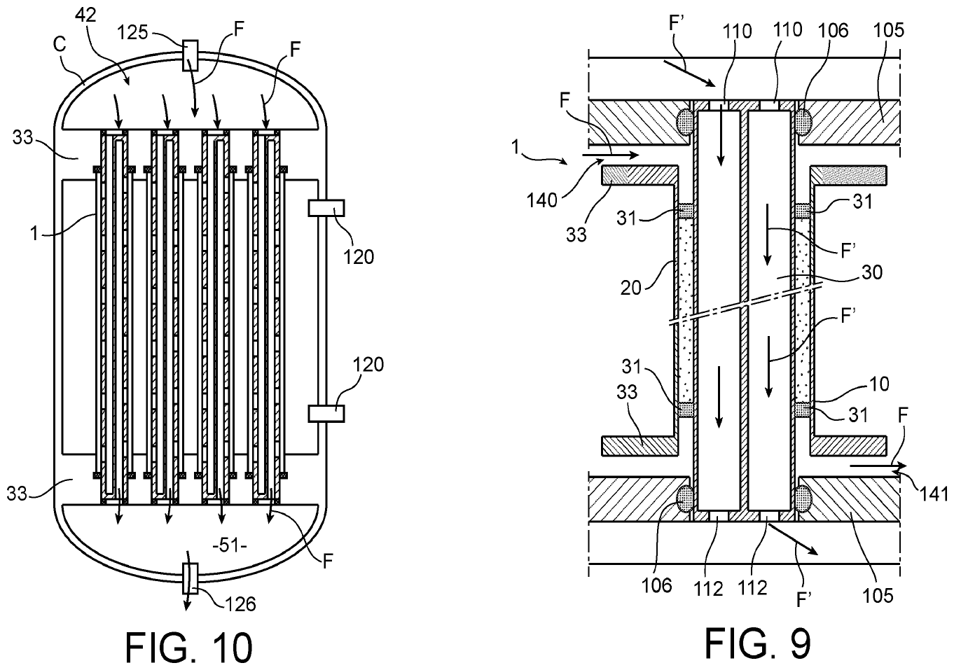

More precisely, as visible in FIG. 7, the supply and extraction of make-up fluid F′ are ensured by the upper and lower ends of the hollow insert 20. The reactive fluids F are then introduced laterally through lateral admission 140 and extraction 141 conduits of reactive fluids, as visible in FIG. 9. The sealed separation between the make-up fluids F′ and the reactive fluids F is ensured by means of separation plates 105, which are removable, introduced into the inlet and outlet plenums, and each disposed around the hollow insert 20. A seal 106 is then placed between each separation plate 105 and the hollow insert 20.

Advantageously, the embodiments of FIGS. 5 and 6 on the one hand, and of FIGS. 7 and 8 on the other hand, allow to produce a tubular plate and tube assembly close to conventional embodiments.

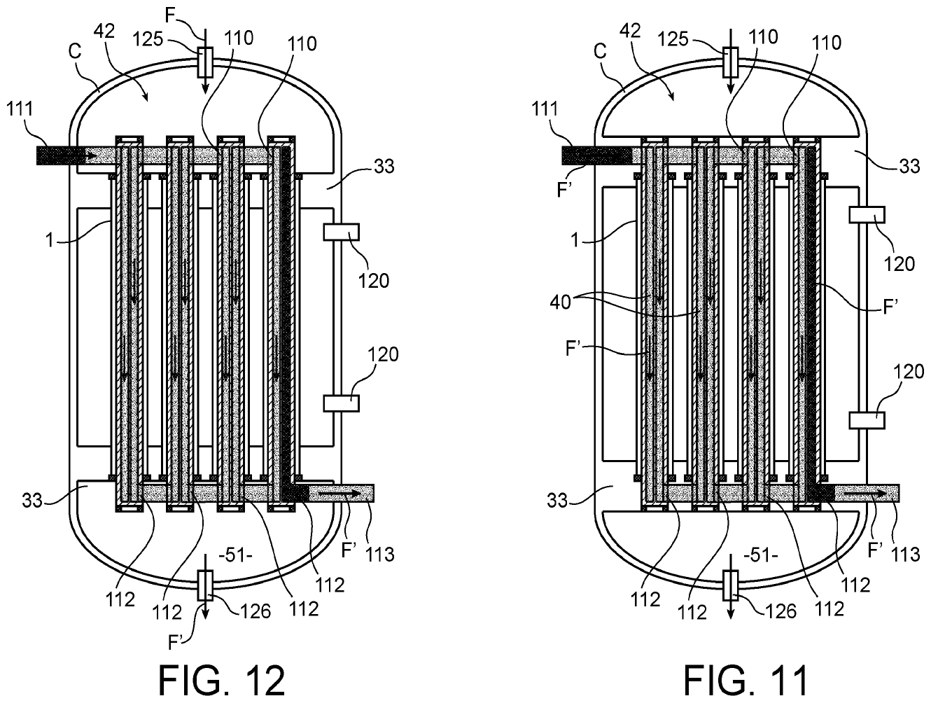

FIGS. 10 and 11 are an illustration of the implementation of a plurality of tubular reactors 1 in accordance with the invention, and in particular according to the variant of FIGS. 1 to 5. Likewise, FIG. 12 is an illustration of the implementation of a plurality of tubular reactors 1 in accordance with the invention according to the variant of FIGS. 6 and 7.

This implementation comprises in particular four tubes 1 disposed parallel to each other in a shell C. The tubular holding plates 33 allow to hold the tubes 1, and to provide a space for the circulation of a heat transfer fluid intended for cooling the tubes 1, via heat transfer fluid supply and evacuation systems 120.

In the example of FIGS. 10 and 11, the distribution chamber(s) 40 are supplied directly from the inlet plenum 42 via reactant supply systems 125. Then, the evacuation of unconverted products and reactants is carried out in the outlet plenum 51 by means of extraction systems 126.

Here, the external tubes are shorter than the inserts 20, and the make-up fluid F′ is distributed directly into the inserts 20 by conduits 111, 113 integrated into the tubular holding plates 33.

In the example of FIG. 12, the hollow inserts 20 are longer. The supply and extraction of the make-up fluid F′ are carried out by a dedicated circuit of conduits 111, 113 located outside the holding plates 33.

The tubular reactor 1 according to the present invention, and in particular the implementation of make-up chambers 100 allowing the circulation of utility or reactive fluids, allows to extend the thermalisation capacities.

This configuration also responds more favourably to the problem of heating of the catalytic powder, particularly in the form of granular catalyst, and thus limits the appearance of hot spots. The result is a more efficient and more durable device. Furthermore, the arrangement of the catalytic powder in the annular space 30 facilitates the cooling of the latter.

### Example of Application

Two digital models were carried out on the principle of the invention with a cooling heat transfer fluid as a make-up fluid.

For said models, a reactant supply at 3 bar and 250° C. was assumed, a homogeneous heat exchange coefficient outside the hollow tube 10 of 1500 W/m²/K with a reference temperature of 250° C., and a release of heat in the porous matrix of 200 W of power.

A first simulation was conducted without internal circulation of heat carrier, and a second simulation with internal circulation of heat carrier, all other things being equal. The results are summarised in Table 1 below. Not only does the internal circulation of a make-up fluid, here a heat transfer fluid, in the auxiliary chambers 100 allows to limit the increase in temperatures, but it also allows for better homogeneity of temperatures in the catalytic bed.

TABLE 1

| | gas | | porous matrix (catalyst) | |
|---|---|---|---|---|
| heat transfer | average | maximum | average | maximum |
| (make-up) | temperature | temperature | temperature | temperature |
| fluid | (° C.) | (° C.) | (° C.) | (° C.) |
| Without | 271 | 292 | 290 | 308 |
| With | 254 | 256 | 271 | 279 |

Of course, the invention is not limited to the exemplary embodiments which have just been described. Various modifications can be made thereto by the person skilled in the art.

In particular, the number, and the respective and angular arrangement, of the make-up 100, collection 50 and distribution 40 chambers may vary.

The direction of circulation of the make-up fluids F′ in the auxiliary chambers 100 can vary.

The choice of the location of the supplies and extractions, in particular at the end of the insert 20 or by lateral tapping of the insert 20, respectively of the make-up fluids F′ and of the reagents F can vary depending on the method.

The supply of the make-up chambers 100 may or may not be associated with the tubular plates 33.

The reactor 1 according to the invention may or may not comprise staged injection means.

For the case where the make-up chambers 100 are used as cooling chambers, as in the previous examples, the efficiency of the system can be further improved by various manipulations aimed at intensifying thermal exchanges and known to the person skilled in the art, such as structuring, in particular micro-structuring, of surfaces, modifications of thermo-hydraulic regimes, among others.

In the case where the hollow insert 20 is the site of an endothermic reaction, the heat supply could be carried out by the heat transfer fluid, instead of the make-up fluid as a cooling fluid.

In particular, the make-up chambers 100 can be used to house a chemical reaction of a nature opposite to that located in the annular space 30, for example a hydrogenation reaction against a dehydrogenation reaction, with the aim of energy integration. This last reaction can for example be considered as taking place through a fixed bed.

Thus, for example, the annular space 30 can be the site of a hydrogenation reaction of the CO₂+4 H₂→CH₄+2H₂O type (ΔH₂₉₈=−165 KJ/mol), and the make-up chambers 100 can be the site of a dehydrogenation reaction on a catalytic fixed bed, based on the GBL/BDO (“butyrolactone/butanediol”) couple of liquid organic hydrogen carrier (LOHC) type molecules of type BDO→GBL+2 H₂ (ΔH₂₉₈=31 KJ/mol of H₂). The reaction enthalpies, and the operating conditions, are compatible so that the energy necessary for dehydrogenation is provided by the hydrogenation reaction.

1. A fixed-bed tubular reactor which extends, along a longitudinal axis, between a first end and a second end, the reactor comprising a bed of catalytic powder confined in an annular space delimited by an external wall of a hollow tube and an internal wall of a hollow insert disposed coaxially in the hollow tube,
the hollow insert comprising at least one distribution chamber and at least one collection chamber, separated from each other by at least one first separating wall, the at least one distribution chamber and the at least one collection chamber comprising, respectively, a gas admission opening at the first end and a gas evacuation opening at the second end,
wherein the reactor further comprises at least one make-up chamber separated from the at least one distribution chamber and from the at least one collection chamber by at least one first separating wall, the at least one make-up chamber comprising an inlet orifice for at least one make-up fluid, distinct from the gases circulating in the at least one distribution chamber and in the at least one collection chamber, at the first end and an outlet orifice of the at least one make-up fluid at the second end.

2. The reactor of claim 1, wherein each of the at least one distribution chamber, the at least one collection chamber, and the at least one make-up chamber is delimited by a section of the internal wall and two first separating walls.

3. The reactor of claim 1, further comprising at the first end and at the second end, respectively, a distributor space and a collector space between which the hollow insert is disposed.

4. The reactor of claim 1, wherein the tube and the insert are held by at least two tubular holding plates respectively at the first and second ends, at least one of the at least two tubular holding plates comprising an inlet or outlet conduit respectively fluidly connected to a make-up fluid inlet or outlet orifice and formed in the at least one of the at least two tubular holding plates to allow lateral admission and/or extraction of make-up fluid.

5. The reactor of claim 4, wherein the axial lengths of the tube and the insert are different, the axial length of the tube being shorter than the axial length of the insert.

6. The reactor of claim 3, wherein the tube and the insert are held by at least two tubular holding plates respectively at the first and second ends, and
wherein at least one inlet or outlet conduit respectively fluidly connected to a make-up fluid inlet or outlet orifice is located respectively in the distributor space and in the collector space, outside the at least two tubular holding plates, to allow lateral admission and/or extraction of make-up fluid.

7. The reactor of claim 3, wherein the tube and the insert are held by at least two tubular holding plates respectively at the first and second ends, and
said wherein the inlet orifice and the outlet orifice of the at least one make-up fluid are respectively formed in the upper and lower ends of the insert to respectively allow a supply from the distributor space and an extraction from the collector space, the reactor further comprising at least one lateral gas supply conduit and at least one lateral gas extraction conduit.

8. The reactor of claim 7, wherein the at least one lateral supply conduit and the supply of make-up fluid to the distributor space on the one hand, and the at least one lateral extraction conduit and the extraction of make-up fluid from the collector space on the other hand, are separated by a sealed separation plate.

9. The reactor of claim 8, wherein a seal is disposed between the insert and each of the sealed separation plates.

10. The reactor of claim 1, wherein the flow of the at least one make-up fluid is carried out in a co-current or counter-current manner to the flow of gases in the at least one distribution and collection chamber.

11. The reactor of claim 1, wherein the catalytic powder is retained in the annular space by a seal made of fibrous material at each of the ends of the annular space.

12. The reactor of claim 1, wherein the at least one make-up fluid is a heat transfer fluid.

13. The reactor of claim 1, wherein the insert is a one-piece part.

14. A method of synthesizing a fuel or a combustible, the method comprising:
implementing an endothermic or exothermic reaction within the fixed-bed tubular reactor of claim 1.

15. The method of claim 14, wherein the annular space allows a hydrogenation reaction, and
wherein the at least one make-up chamber allows a dehydrogenation reaction, or vice versa.
